(19) United States
(12) Patent Application Publication
(10) Pub. No.: US 2026/0069854 A1
(43) Pub. Date: Mar. 12, 2026

(54) METHOD AND DEVICE FOR INTELLIGENT INFORMATION MANAGEMENT OF IMPLANTABLE LEFT VENTRICULAR ASSIST DEVICE

(71) Applicant: BRIOHEALTH TECHNOLOGIES, INC., Jiangsu (CN)

(72) Inventors: Jinjing MIAO, Suzhou (CN); Xinxin ZHENG, Suzhou (CN); Yongjun YIN, Suzhou (CN); Wenfeng XU, Suzhou (CN)

(73) Assignee: BRIOHEALTH TECHNOLOGIES, INC., Jiangsu (CN)

(21) Appl. No.: 19/132,516

(22) PCT Filed: Feb. 21, 2024
(86) PCT No.: PCT/CN2024/077896

§ 371 Date: May 23, 2025

(30) Foreign Application Priority Data
Jan. 5, 2024 (CN) 202410016946.0

(51) Int. Cl. A61M 60/592; A61M 60/17

(52) U.S. Cl. CPC A61M 60/592; A61M 60/17; A61M 2205/18; A61M 2205/3553; A61M 2205/502; A61M 2205/52

(57) ABSTRACT

A communication module is disclosed. The communication module is connected to a patient controller of an implantable left ventricular assist device and a cloud device. The implantable left ventricular assist device further includes a blood pump connected to the patient controller. The communication module is configured to receive the present operating data of the blood pump transmitted by the patient controller. The communication module is further configured to periodically publish the present operating data to the cloud device. An implantable left ventricular assist system is also disclosed. The system includes an implantable left ventricular assist device, a cloud device, a mobile device, and the communication module. A method for intelligent information management of an implantable left ventricular assist device applied to the communication module is further disclosed.

### CROSS-REFERENCE TO RELATED APPLICATIONS

The present application claims priority to Chinese patent application No. 202410016946.0, filed on Jan. 5, 2024, the entire content of which is incorporated herein by reference.

### TECHNICAL FIELD

The present disclosure relates to the field of medical device technologies, and in particular, to a communication module, an implantable left ventricular assist system, and a method for intelligent information management of an implantable left ventricular assist device.

### BACKGROUND

With the development of medical device technology, implantable left ventricular assist devices have emerged to provide hemodynamic support to patients. The implantable left ventricular assist device includes a blood pump implanted in a human body, a patient controller, a power source, and a medical monitor. The patient controller is connected to the blood pump, and the medical monitor communicates with the patient controller through a serial port. The medical workers can use the medical monitor to monitor a real-time state of the blood pump to monitor a real-time state of a patient.

The medical monitor is unable to monitor the implantable left ventricular assist device in time after the patient is discharged from the hospital. The present implantable left ventricular assist device has the problem of low safety in remote real-time monitoring scenarios.

### SUMMARY

It is necessary to provide a communication module, an implantable left ventricular assist system, and a method for intelligent information management of an implantable left ventricular assist device to improve safety in response to the above technical problem.

In a first aspect, the present disclosure provides a communication module configured to be connected to a patient controller of an implantable left ventricular assist device and a cloud device. The implantable left ventricular assist device further includes a blood pump connected to the patient controller. The communication module is configured to receive present operating data of the blood pump transmitted by the patient controller. The communication module is configured to periodically publish the present operating data to the cloud device.

In an embodiment, the communication module is configured to receive present power data of the implantable left ventricular assist device transmitted by the patient controller. The communication module is configured to periodically publish the present power data to the cloud device.

In an embodiment, the communication module is configured to receive a device alarm message of the implantable left ventricular assist device transmitted by the patient controller. The communication module is configured to periodically publish the device alarm message to the cloud device.

In an embodiment, the communication module includes: a communication unit configured to be connected to the patient controller, a power source of the implantable left ventricular assist device, and the cloud device respectively; a display unit; and a buck unit configured to be connected to the patient controller and the power source respectively, and also configured to be connected to the communication unit and the display unit respectively.

In an embodiment, the communication module includes: a power interface configured to be connected to the power source; and a plurality of cables connected between the patient controller and the power interface. The plurality of cables include a monitoring device communication cable, a battery communication cable, a power cable, and a grounding cable. The buck unit is connected to the power cable and the grounding cable respectively, and the communication unit is connected to the monitoring device communication cable.

In a second aspect, the present disclosure provides an implantable left ventricular assist system, including: a communication module; an implantable left ventricular assist device including a patient controller, and a blood pump connected to the patient controller, the patient controller being connected to a communication module, configured to acquire present operating data of the blood pump, and transmit the acquired present operating data to the communication module; a cloud device connected to the communication module; and a mobile device connected to the cloud device. The communication module is configured to periodically publish the acquired present operating data to the cloud device, and the cloud device is configured to store the acquired present operating data and provide data access to the mobile device.

In an embodiment, the patient controller is configured to acquire present power data of the implantable left ventricular assist device and transmit the present power data to the communication module. The communication module is configured to receive the present power data and periodically publish the present power data to the cloud device. The cloud device is configured to transmit the present power data to the mobile device in response to receiving the present power data. The mobile device is configured to output a corresponding prompt message in response to receiving the present power data.

In an embodiment, the communication module is configured to receive a device alarm message of the implantable left ventricular assist device transmitted by the patient controller and periodically publish the device alarm message to the cloud device. The cloud device is configured to transmit the device alarm message to the mobile device in response to receiving the device alarm message. The mobile device is configured to output a corresponding prompt message in response to receiving the device alarm message.

In an embodiment, the communication module includes: a communication unit connected to the patient controller and the cloud device, respectively, and further connected to a power source of the implantable left ventricular assist device; a display unit; and a buck unit connected to the patient controller, the communication unit, and the display unit, respectively, and further connected to the power source.

In an embodiment, the communication module includes: a power interface configured to be connected to the power source; and a plurality of cables connected between the patient controller and the power interface. The plurality of cables include a monitoring device communication cable, a battery communication cable, a power cable, and a grounding cable. The buck unit is connected to the power cable and the grounding cable, respectively, and the communication unit is connected to the monitoring device communication cable.

In a third aspect, the present disclosure provides a method for intelligent information management of an implantable left ventricular assist device. The method is applied to the communication module described above. The method includes: receiving, by the communication module, present device data transmitted by the patient controller, the present device data including at least one of the present operating data of the blood pump, the present power data of the implantable left ventricular assist device, or the device alarm message of the implantable left ventricular assist device; and publishing, by the communication module, the present device data to the cloud devices periodically.

In an embodiment, the present operating data includes present start-stop state data and present pump speed data of the blood pump.

In an embodiment, the publishing, by the communication module, the present device data to the cloud device periodically includes: establishing, by the communication module, a MQTT connection to the cloud device in response to the communication module being connected to an access point; sending, by the communication module, a subscription request to the cloud device if the MQTT connection is successfully established, the subscription request including the MQTT subscription topic; and publishing, by the communication module, the present device data to the cloud device periodically if the MQTT subscription topic is successfully subscribed and no MQTT subscription information is received from the cloud device.

In an embodiment, the method further includes: executing, by the communication module, a corresponding request based on the MQTT subscription information, if the MQTT subscription topic is successfully subscribed and the MQTT subscription information is received from the cloud device.

In an embodiment, the method further includes: publishing, by the communication module, historical device data to the cloud device if the MQTT subscription information includes a data export request; and reporting, by the communication module, an export result to the cloud device after the historical device data is published.

Details of one or more embodiments of the present disclosure are set forth in the accompanying drawings and description below. Other features, objects, and advantages of the present disclosure will become apparent from the specification, the accompanying drawings, and the claims.

### BRIEF DESCRIPTION OF THE DRAWINGS

In order to more clearly illustrate the technical solutions in the embodiments of the present disclosure or the related technology, the following will briefly introduce the accompanying drawings that need to be used in the description of the embodiments or the related technology, and it is apparent that the accompanying drawings in the following description are only some of the embodiments of the present disclosure, and for the person of ordinary skill in the field, other attachments can be obtained according to these attachments without putting in creative labor.

FIG. 1 is a block diagram illustrating a configuration of the implantable left ventricular assist device according to some embodiments.

FIG. 2 is a block diagram illustrating a configuration of the communication module according to some embodiments.

FIG. 3 is a block diagram illustrating the configuration of the communication module according to some other embodiments.

FIG. 4 is a block diagram illustrating the configuration of the communication module according to some further embodiments.

FIG. 5 is a block diagram illustrating a configuration of an implantable left ventricular assist system according to some embodiments.

FIG. 6 is a flow diagram illustrating processes of intelligent information management of the cloud device according to some embodiments.

FIG. 7 is a flow diagram illustrating processes of intelligent information management of the cloud device according to some other embodiments.

FIG. 8 is a schematic diagram illustrating an internal configuration of the cloud device according to some embodiments.

FIG. 9 is a schematic diagram illustrating an internal configuration of the mobile device according to some embodiments.

FIG. 10 is a flow diagram illustrating a method for intelligent information management of the implantable left ventricular assist device according to some embodiments.

FIG. 11 is a flow diagram illustrating steps of intelligent information management of the implantable left ventricular assist device according to some embodiments.

FIG. 12 is a flow diagram illustrating a method for intelligent information management of the implantable left ventricular assist device according to some other embodiments.

FIG. 13 is a flow diagram illustrating processes of intelligent information management for the communication module according to some embodiments.

### DETAILED DESCRIPTION OF THE EMBODIMENTS

In order to make the above objectives, features and advantages of the present disclosure more clear and understandable, specific implementations of the present disclosure are described in detail below with reference to the accompanying drawings. In the following description, many specific details are set forth in order to fully understand the present disclosure. However, the present disclosure can be implemented in many other ways different from those described herein, and those skilled in the art can make similar improvements without departing from the connotation of the present disclosure. Therefore, the present disclosure is not limited by the specific embodiments disclosed below.

In the description of the present disclosure, it should be understood that the orientation or position relationship indicated by the terms “center”, “longitudinal”, “transverse”, “length”, “width”, “thickness”. “upper”, “lower”, “front”, “back”, “left”, “right”, “vertical”, “horizontal”, “top”, “bottom”, “inner”, “outer”, “clockwise”, “counterclockwise”, “axial”, “radial”, “circumferential”, etc. are based on the orientation or position relationship shown in the accompanying drawings and are merely intended to facilitate the description of the present disclosure and simplify the description, rather than indicating or implying that the indicated device or element must have a particular orientation or be constructed and operated in a particular orientation, and therefore are not to be interpreted as limiting the present disclosure.

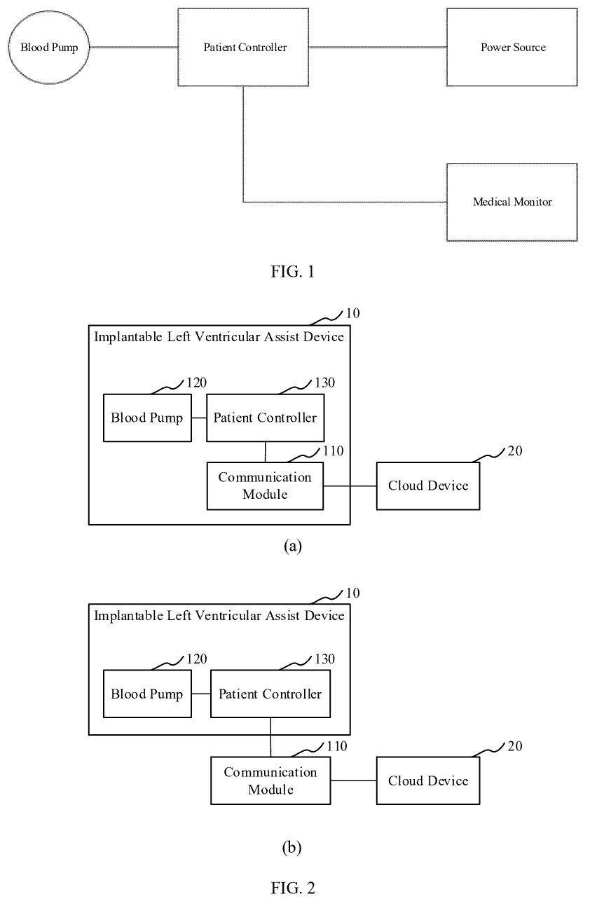

The implantable left ventricular assist device can provide hemodynamic support for patients with heart failure. As shown in FIG. 1, the implantable left ventricular assist device includes a blood pump implanted in a human body, a patient controller, a power source such as an external battery or adapter, and a medical monitor, among other things. The patient controller is connected to the blood pump through a percutaneous cable, and the patient controller is connected to the power source to power the entire implantable left ventricular assist device. Further, in a hospital scenario, a medical monitor can communicate with the patient controller through a serial port. The patient controller is a controller provided for the patient and can display state information of both the blood pump and the power source, and other state information in real time. The medical workers can use the medical monitor to monitor and control the implantable left ventricular assist device.

In the above solution, there is still a gap for remote monitoring of the real-time state of the patient after the patient is discharged from the hospital. When the implantable left ventricular assist device has an abnormal running, the medical workers cannot be notified instantly, and the first aid treatment is required depending on the patient. There is no doubt that there is an unforeseeable risk to the patient if a special abnormality occurs and cannot be resolved by the patient. Meanwhile, the growing number of patients will undoubtedly pose a huge challenge to clinical support and after-sales management. The operational safety of conventional implantable left ventricular assist devices needs to be improved, both from the point of view of patient risk control and after-sales service tracking.

In an embodiment, as shown in FIG. 2, a communication module 110 is provided. The communication module 110 is configured to be connected to a patient controller 130 of the implantable left ventricular assist device 10. The implantable left ventricular assist device 10 further includes a blood pump 120 connected to the patient controller 130. The communication module 110 is further configured to be connected to the cloud device 20. The communication module 110 receives present operating data of the blood pump 120 transmitted by the patient controller 130. The communication module 110 periodically uploads the present operating data to the cloud device 20.

Specifically, the blood pump 120 can be implanted in the left ventricle of the heart, the patient controller 130 can be arranged outside a body of the patient, and the patient controller 130 is connected to the blood pump 120, and can acquire present operating data of the blood pump 120 in real time to monitor a real-time state of the blood pump 120. The operating data of the blood pump 120 can include data such as a pump speed, a flow rate, and a power consumption of the blood pump 120. The communication module 110 is connected to the patient controller 130 and can receive the present operating data of the blood pump 120 transmitted by the patient controller 130. As shown in figure (a) in FIG. 2, the communication module 110 can be integrated into the implantable left ventricular assist device 10, e.g., can be integrated inside the patient controller 130. As shown in figure (b) in FIG. 2, the communication module 110 can be independent from the implantable left ventricular assist device 10, and can exchange data with the patient controller 130 wirelessly. The communication module 110 can communicate with the cloud device 20 wirelessly. In the case where the communication module 110 receives the present operating data of the blood pump 120 transmitted by the patient controller 130, the communication module 110 can periodically publish the present operating data to the cloud device 20 to achieve synchronization of the present operating data between the cloud device 20 and the patient controller 130.

In some examples, the patient controller 130 can be connected to the blood pump 120 through a percutaneous cable. The patient controller 130 can also be configured to be connected to the monitoring device, and the medical workers can monitor and control the implantable left ventricular assist device 10 through the monitoring device. For example, the monitoring device can monitor the real-time state of the blood pump 120, and control the start-stop, the pump speed, the Hematocrit (HCT) value, the low-flow threshold, and the high-power threshold of the blood pump 120. The cloud device 20 can be connected to one or more mobile devices and can synchronize the present operating data to the corresponding mobile device for remote monitoring of the implantable left ventricular assist device 10.

Embodiments of the present disclosure provide a communication module 110. The communication module 110 is configured to be connected to a patient controller 130 of the implantable left ventricular assist device 10. The implantable left ventricular assist device 10 further includes a blood pump 120 connected to the patient controller 130. The communication module 110 is configured to be connected to a cloud device 20. The communication module 110 receives the present operating data of the blood pump transmitted by the patient controller 130. The communication module 110 periodically publishes the present operating data to the cloud device 20. By means of the above communication module, the running state of the blood pump 120 can be remotely monitored. After the patient has been discharged from the hospital, in the event of an abnormal running of the implantable left ventricular assist device 10, the medical workers can obtain a corresponding notification from the cloud device in time. As a result, the safety of the implantable left ventricular assist device 10 is improved.

In an embodiment, the present operating data includes present start-stop state data and present pump speed data of the blood pump 120.

Specifically, the present start-stop state data of the blood pump 120 can be used to indicate that the blood pump 120 is presently in a start state or a stop state. The present pump speed data of the blood pump 120 can be used to indicate the real-time pump speed and the target pump speed of the blood pump 120.

In an embodiment, the communication module 110 receives the present power data of the implantable left ventricular assist device 10 transmitted by the patient controller 130.

The communication module 110 periodically publishes the present power data to the cloud device 20.

Specifically, the implantable left ventricular assist device 10 can be connected to a power source for normal running. The power source can be an external power source or an internal power source, and the external power source can include an external battery or an external adapter. The communication module 110 can communicate wirelessly with the cloud device 20. If the communication module 110 receives the present power data of the blood pump 120 transmitted by the patient controller 130, the communication module 110 can periodically publish the present power data to the cloud device 20 such that the present power data is synchronized between the cloud device 20 and the patient controller 130. The present power data of the implantable left ventricular assist device 10 can be information related to the power source presently in use, including power source voltage, current, battery capacity, and the like. The present power data can be used to monitor the power state of the implantable left ventricular assist device 10 to ensure normal running of the implantable left ventricular assist device 10.

In some examples, the communication module 110 can be arrange between the patient controller 130 and the power source. The communication module 110 is connected to the patient controller 130 and the power source, respectively. The communication module 110 can be integrated within the power source, and the power source can be arranged outside the body.

In an embodiment, the communication module 110 receives a device alarm message of the implantable left ventricular assist device 10 transmitted by the patient controller 130. The communication module 110 periodically publishes the device alarm message to the cloud device 20.

Specifically, the communication module 110 can communicate wirelessly with the cloud device 20. In the case where the communication module 110 receives the device alarm message of the implantable left ventricular assist device 10 transmitted by the patient controller 130, the communication module 110 can periodically publish the device alarm message to the cloud device 20 to achieve synchronization of the device alarm message between the cloud device 20 and the patient controller 130. The device alarm message of the implantable left ventricular assist device 10 can be information related to abnormal or faulty conditions detected during the running of the implantable left ventricular assist device 10 and notified to a user or a medical worker by means of an alarm. The device alarm message can be used to alert the user or the medical worker of the abnormal condition of the implantable left ventricular assist device 10 to take appropriate measures to deal with it.

In an embodiment, the communication module 110 includes a communication unit 112, a display unit 114, and a buck unit 116. The communication unit 112 is configured to be connected to the patient controller 130 and the power source of the implantable left ventricular assist device 10, respectively. The communication unit 112 is further configured to be connected to the cloud device 20. The buck unit 116 is configured to be connected to the patient controller 130 and the power source, respectively. The buck unit 116 is also connected to the communication unit 112 and the display unit 114, respectively.

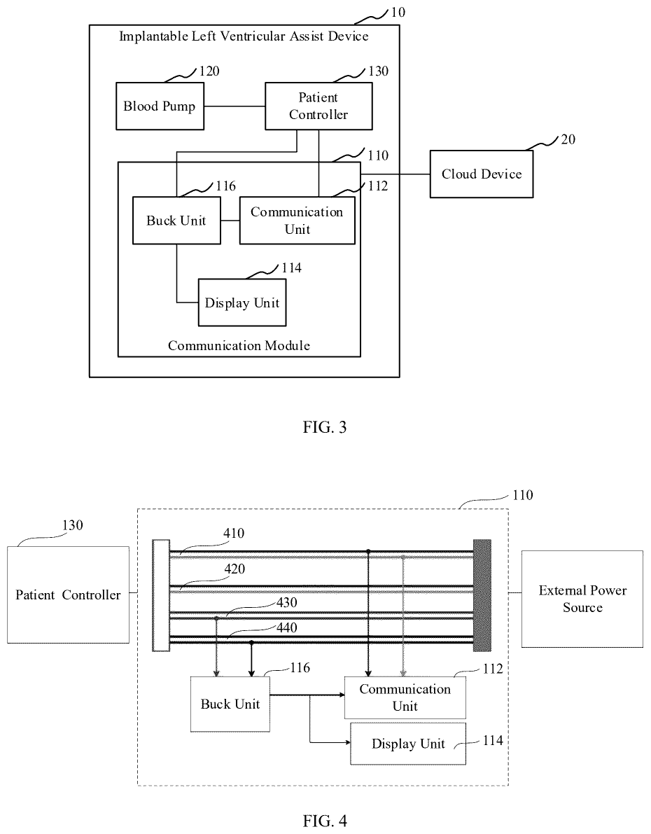

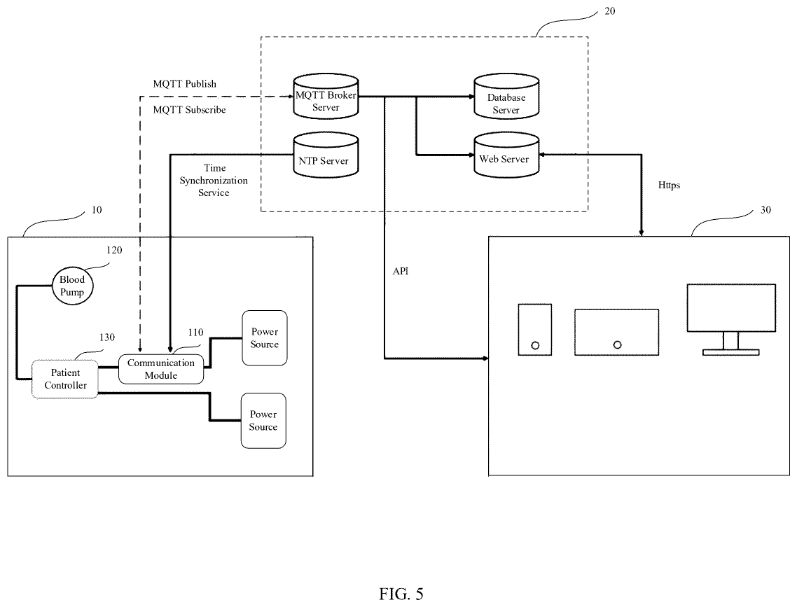

Specifically, as shown in FIG. 3, taking the communication module 110 integrated in the implantable left ventricular assist device 10 as an example, the communication unit 112 can be connected to the patient controller 130 to obtain the present operating data of the blood pump 120, the present power data of the implantable left ventricular assist device 10, and/or the device alarm message transmitted by the patient controller 130. The buck unit 116 can be connected to the patient controller 130, the communication unit 112, and the display unit 114. The buck unit 116 can also be connected to an external power source to enable step-down power supply to the patient controller 130, the communication unit 112, and the display unit 114. The communication unit 112 can be configured to be connected to the cloud device 20 and communicate wirelessly with the cloud device 20. If the communication unit 112 receives the present operating data of the blood pump 120 transmitted by the patient controller 130, the communication unit 112 can periodically publish the present operating data to the cloud device 20 to achieve synchronization of the present operating data between the cloud device 20 and the patient controller 130. In the case where the communication unit 112 receives the present power data of the blood pump 120 transmitted by the patient controller 130, the communication unit 112 can periodically publish the present power data to the cloud device 20, achieving synchronization of the present power data between the cloud device 20 and the patient controller 130. In the case where the communication unit 112 receives the device alarm message of the implantable left ventricular assist device 10 transmitted by the patient controller 130, the communication unit 112 can periodically publish the device alarm message to the cloud device 20, realizing synchronization of the device alarm message between the cloud device 20 and the patient controller 130. The display unit 114 can display the present operating data, the present power data of the implantable left ventricular assist device 10, and/or the device alarm message. The present operating data includes the present start-stop state data and the present pump speed data of the blood pump 120 In an embodiment, as shown in FIG. 4, the communication module 110 includes a power interface configured to be connected to the power source. The communication module 110 further includes a plurality of cables connected between the patient controller 130 and the power interface. The cables include a monitoring device communication cable 410, a battery communication cable 420, a power cable 430, and a grounding cable 440. The buck unit 116 is connected to the power cable 430 and the grounding cable 440, respectively. The communication unit 112 is connected to the monitoring device communication cable 410.

Specifically, the communication module 110 can be connected in series between the patient controller 130 and an external power source such as a battery or adapter. The plurality of cables of the communication module 110 can all be connected in a hardwired manner between the patient controller 130 and the external power source. The buck unit 116 is connected to the power cable 430 and the grounding cable 440, respectively. After performing the DC-DC (direct current to direct current) buck conversion of the external power source, the buck unit 116 supplies power to the communication unit 112 and the display unit 114 respectively, both of which are connected to the buck unit 116. Further, in the event of a failure of the communication module 110, the patient controller 130 and the external power source connected to the communication module 110 cannot be damaged, thereby improving the safety and reliability of the communication module 110.

In some examples, the communication module 110 can be connected in series between a power cable connector of the patient controller 130 and an external power source. The monitoring device communication cable 410 can include a data transmission line and a data reception line. The battery communication cable 420 can include a clock line and a data line. The communication unit 112 can support at least one of Wi-Fi (wireless network communication technology) communication, 4G (fourth generation mobile communication technology) communication, or 5G (fifth generation mobile communication technology) communication. The display unit 114 can include buttons and an LED (light emitting diode) display.

In an embodiment, as shown in FIG. 5, an implantable left ventricular assist system is provided. The implantable left ventricular assist system includes an implantable left ventricular assist device 10, a cloud device 20, a mobile device 30, and a communication module 110.

The implantable left ventricular assist device 10 includes a patient controller 130, and a blood pump 120 connected to the patient controller 130. the patient controller 130 is connected to a communication module 110, and the patient controller 130 acquires the present operating data of the blood pump 120 and transmits the present operating data to the communication module 110. The communication module 110 is connected to the cloud device 20. The communication module 110 periodically publishes the present operating data to the cloud device 20. The cloud device 20 is connected to the mobile device 30, and the cloud device 20 stores the present operating data acquired and provides data access to the mobile device 30.

Specifically, the communication module 110 of the implantable left ventricular assist device 10 can be a central hub for the acquisition of the present operating data, and uploading of the present operating data to the cloud device 20, which can serve as a medical monitor to monitor the operating state of the blood pump 120. For the cloud device 20, the communication module 110 corresponds to a MQTT client. In the implantable left ventricular assist system, the implantable left ventricular assist device 10 is located on the device side, the cloud device 20 is located on the cloud side, and the mobile device 30 is located on the mobile side. The cloud device 20 can be a server or a cluster of servers that can be shared by both the device side and the mobile side, and can be accessed through a fixed public IP address. The cloud device 20 can provide services of a MQTT broker server, a database server, a network time protocol (NTP) server, and a web server. The cloud device 20 can be a proxy for MQTT communication, responsible for data subscription and publication on the device side and the mobile side, and protects the security and integrity of the data through SSL (Secure Sockets Layer)/TSL (Transport Layer Security) encryption and authentication. The cloud device 20 can be a database server that can provide reliable data storage and access services, for example, storage and access services for present operating data, historical operating data, and historical operation data of the mobile side. The cloud device 20 as a network time protocol server can provide a time synchronization service. The cloud device 20 as a web server can provide front-end interaction service for the mobile side, for example, a web interactive service and a web socket service. The mobile side can include one or more mobile devices 30. The mobile device 30 is a mobile, multi-platform device with the ability to communicate with the cloud and can serve healthcare, clinical, and after-sales services. The mobile side can support remote export of historical operating data from the cloud device 20, for example, by sending a data export request to the cloud device 20. Remote monitoring of the real-time state of the patient can be realized through the mobile side.

With the above system, remote monitoring of the operating state of the blood pump 120 can be realized, and after the patient has been discharged from the hospital, the medical workers will also be able to obtain appropriate notifications from the mobile device 30 in time in the event of abnormalities in the running of the implantable left ventricular assist device 10, which improves the safety of the implantable left ventricular assist device 10.

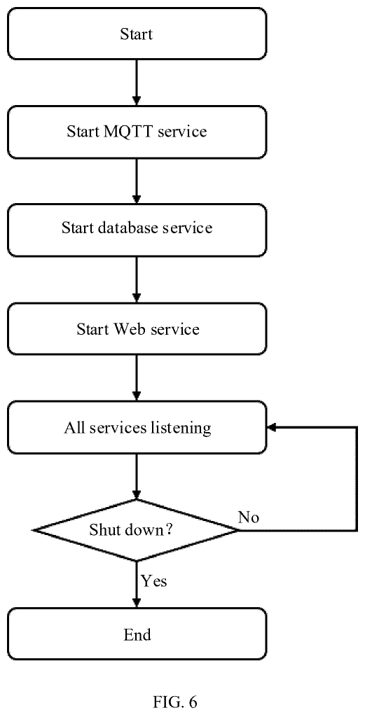

In some examples, the cloud device 20 can be connected to one or more mobile devices 30, and one or more implantable left ventricular assist devices 10. The mobile device 30 can include at least one of a cell phone, a tablet, or a personal computer. As shown in FIG. 6, after the cloud device 20 is started, the cloud device 20 can start the MQTT service, the database service, and the web service in turn, and keep all services in a listening state. It can determine whether the cloud device 20 needs to be shut down, and if so, the cloud device 20 ends its running; if not, return to the step of keeping all services in a listening state.

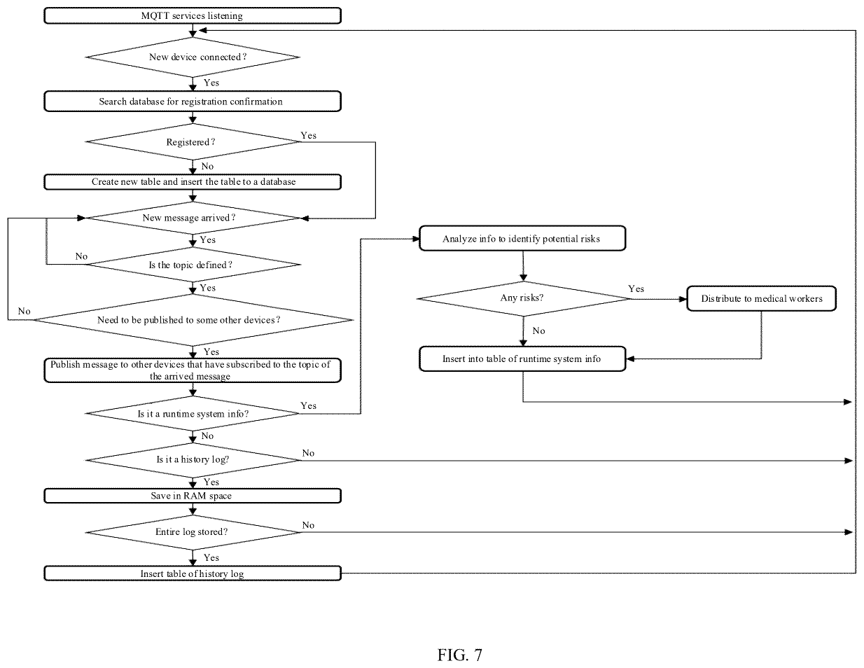

In some examples, as shown in FIG. 7, during the MQTT service listening process, the cloud device 20 detects whether a new device is connected, and if so, searches the database to confirm whether the new device is registered. If the new device is not registered, a new table is created for the new device and inserted into the database. If the new device has been registered, the cloud device 20 detects whether a new message has arrived. If the cloud device 20 detects the arrived message, the cloud device 20 determines whether the topic corresponding to the arrived message has been defined. If the topic of the arrived message has been defined, the cloud device 20 determines whether it is necessary to publish the arrived message to some other devices. The cloud device 20 can publish the arrived message to the other devices that have subscribed to the topic of the arrived message. The cloud device 20 can further determine whether the arrived message is system runtime information. If the arrived message is the system runtime information, the cloud device 20 can analyze whether there is a potential risk in the system runtime information. If there is a potential risk, the cloud device 20 can distribute the system running time information to the medical worker. If there is no potential risk, the cloud device 20 can insert the system running time information into the system running time information table. In the case that the cloud device 20 determines that the arrived message is not the system running time information, the cloud device 20 can determine whether the arrived message is a history log. If the arrived message is a history log, the cloud device 20 can store the history log to the memory space. If all of the history logs are stored, the cloud device 20 can insert the history logs into the history log table.

In some examples, if the mobile device 30 is configured with an APP as a client, in the event of an abnormality of the implantable left ventricular assist device 10, a corresponding push application function program can be subscribed through the APP program interface to receive device alarm message and prompt the medical worker with information about the corresponding recommended measures. If the mobile device 30 is configured with a webpage as a client, the presently online implantable left ventricular assist device 10 can be viewed through the webpage, and the real-time state of each implantable left ventricular assist device 10 can be monitored. The mobile device 30 can generate a data export request through the webpage to enable remote export of historical operating data. The mobile device 30 can also carry out an analysis based on the big data of the users on the mobile side through the webpage, which can be used to provide a warning alert for the user with a view to avoiding risks as far as possible.

In an embodiment, the patient controller 130 acquires the present power data of the implantable left ventricular assist device 10 and transmits the present power data to the communication module 110. The communication module 110 receives the present power data and periodically publishes the present power data to the cloud device 20. The cloud device 20 transmits the present power data to the mobile device 30 in response to receiving the present power data. The mobile device 30 outputs a corresponding prompt message in response to receiving the present power data.

Specifically, the communication module 110 of the implantable left ventricular assist device 10 can be a central hub for uploading the present power data to the cloud device 20, and periodically publishing the present power data to the cloud device 20 for remote monitoring of the power state of the implantable left ventricular assist device 10. For example, the cloud device 20 can transmit the received the present power data to the mobile device 30, and the user can remotely monitor the power state of the implantable left ventricular assist device 10 by using the mobile device 30. The cloud device 20, as a database server, can provide a reliable data storage and access service, for example, a storage and access service for the present power data as well as the historical power data. The mobile side can support remote export of the historical power data in the cloud device 20, for example, by sending a data export request to the cloud device 20.

In an embodiment, the communication module 110 receives the device alarm message of the implantable left ventricular assist device 10 transmitted by the patient controller 130 and periodically publishes the device alarm message to the cloud device 20. The cloud device 20 transmits the device alarm message to the mobile device 30 in response to receiving the device alarm message. The mobile device 30 outputs a corresponding prompt message in response to receiving the device alarm message.

Specifically, if the cloud device 20 receives the device alarm message of the implantable left ventricular assist device 10, the cloud device 20 can transmit the device alarm message to the mobile device 30. In response to receiving the device alarm message, the mobile device 30 outputs the corresponding prompt message to prompt the medical workers to carry out the corresponding processing of the implantable left ventricular assist device 10 for remote monitoring of the real-time state of the patient.

Specifically, the communication module 110 of the implantable left ventricular assist device 10 can be a central hub for uploading the device alarm message to the cloud device 20. The communication module 110 periodically publishes the device alarm message to the cloud device 20 for remote monitoring of the implantable left ventricular assist device 10. For example, the cloud device 20 can transmit the received the device alarm message to the mobile device 30, and the user can remotely monitor the implantable left ventricular assist device 10 by using the mobile device 30. In the event of an abnormality in the implantable left ventricular assist device 10, the mobile device 30 can obtain the device alarm message in time, and can prompt the medical worker with information on corresponding recommended measures.

In an embodiment, the communication module 110 includes a communication unit 112, a display unit 114, and a buck unit 116. The communication unit 112 is connected to the patient controller 130 and the cloud device 20, respectively, and is further connected to the power source of the implantable left ventricular assist device 10. The buck unit 116 is connected to the patient controller 130, the communication unit 112, and the display unit 114, respectively. The buck unit 116 is further configured to be connected to the power source.

Specifically, the communication unit 112 can be connected to the patient controller 130 to obtain the present operating data of the blood pump 120, the present power data of the implantable left ventricular assist device 10, and/or the device alarm message transmitted by the patient controller 130. The buck unit 116 can be connected to the patient controller 130, the communication unit 112, and the display unit 114. The buck unit 116 can be connected to an external power source to enable step-down power supply to the patient controller 130, the communication unit 112, and the display unit 114. The communication unit 112 can be configured to be connected to the cloud device 20 and communicate wirelessly with the cloud device 20. If the communication unit 112 receives the present operating data of the blood pump 120 transmitted by the patient controller 130, the communication unit 112 can periodically publish the present operating data to the cloud device 20 to achieve synchronization of the present operating data between the cloud device 20 and the patient controller 130. If the communication unit 112 receives the present power data of the blood pump 120 transmitted by the patient controller 130, the communication unit 112 can periodically publish the present power data to the cloud device 20 to achieve synchronization of the present power data between the cloud device 20 and the patient controller 130. If the communication unit 112 receives the device alarm message of the implantable left ventricular assist device 10 transmitted by the patient controller 130, the communication unit 112 can periodically publish the device alarm message to the cloud device 20 to realize synchronization of the device alarm message between the cloud device 20 and the patient controller 130. The display unit 114 can display present operating data, the present power data of the implantable left ventricular assist device 10, and/or the device alarm message. The present operating data includes present start-stop state data and present pump speed data of the blood pump 120.

In an embodiment, the communication module 110 includes a power interface configured to be connected to the power source. The communication module 110 further includes a plurality of cables connected between the patient controller 130 and the power interface. The cables include a monitoring device communication cable 410, a battery communication cable 420, a power cable 430, and a grounding cable 440. The buck unit is connected to the power cable 430 and the grounding cable 440, respectively. The communication unit is connected to the monitoring device communication cable 410.

Specifically, the communication module 110 can be connected in series between the patient controller 130 and an external power source such as a battery or adapter. The plurality of cables of the communication module 110 can all be connected in a hardwired manner between the patient controller 130 and the external power source. The buck unit 116 is connected to the power cable 430 and the grounding cable 440, respectively. After DC-DC (direct present-direct present) buck conversion of the external power source, the buck unit 116 supplies power to the communication unit 112 and the display unit 114 respectively, both of which are connected to the buck unit 116. Further, in the event of a failure of the communication module 110, the patient controller 130 and the external power source connected to the communication module 110 cannot be damaged, thereby improving the safety and reliability of the communication module 110.

In some examples, the communication module 110 can be connected in series between a power cable connector of the patient controller 130 and an external power source. The monitoring device communication cable 410 can include a data transmission line and a data reception line. The battery communication cable 420 can include a clock line and a data line. The communication unit 112 can support at least one of Wi-Fi (wireless network communication technology) communication, 4G (fourth generation mobile communication technology) communication, or 5G (fifth generation mobile communication technology) communication. The display unit 114 can include buttons and an LED (light emitting diode) display.

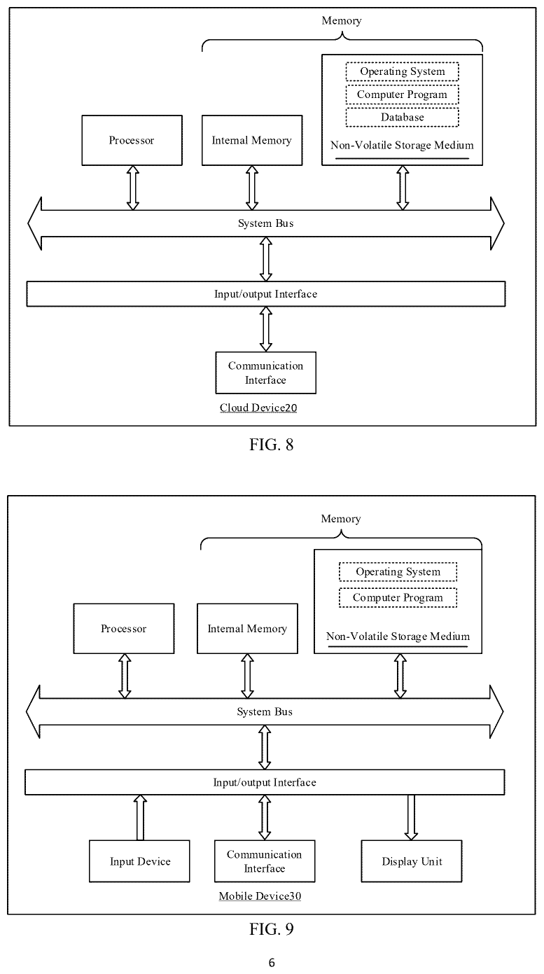

In an exemplary embodiment, a cloud device 20 is provided, which can be a server, and an internal structure diagram of which can be shown in FIG. 8. The cloud device 20 includes a processor, a memory, an input/output interface (I/O), and a communication interface. The processor, the memory and the input/output interface are connected through a system bus, and the communication interface is connected to the system bus through the input/output interface. The processor of the cloud device 20 is used to provide computing and control capabilities. The memory of this cloud device 20 includes a non-volatile storage medium and an internal memory. The non-volatile storage medium stores an operating system, a computer program, and a database. The internal memory provides an environment for the running of the operating system and the computer program in the non-volatile storage medium. The database of this cloud device 20 is used to store present device data, historical device data, and historical operation data of the mobile side. The input/output interface of this cloud device 20 is used to exchange information between the processor and an external device. The communication interface of the cloud device 20 is used to communicate with an external side through a network connection.

In an exemplary embodiment, a mobile device 30 is provided, which can be a terminal. FIG. 9 shows an internal configuration of the mobile device 30. The mobile device 30 includes a processor, a memory, an input/output interface, a communication interface, a display unit, and an input device. The processor, the memory and the input/output interface are connected through a system bus, and the communication interface, the display unit and the input device are connected to the system bus through the input/output interface. The processor of the mobile device 30 is used to provide computing and control capabilities. The memory of the mobile device 30 includes a non-volatile storage medium and an internal memory. The non-volatile storage medium stores an operating system and a computer program. The internal memory provides an environment for the running of the operating system and computer program in the non-volatile storage medium. The input/output interface of the mobile device 30 is used to exchange information between the processor and an external device. The communication interface of the mobile device 30 is used for communicating with an external side in a wired or wireless manner, the wireless manner being realized by WIFI, mobile cellular network, NFC (Near Field Communication) or other technologies. The display unit of the mobile device 30 is used to form a visually visible picture, which can be a display screen, a projection device or a virtual reality imaging device. The display can be an LCD or an e-ink display, and the input unit of the mobile device 30 can be a touch layer covered on the display, a button, a trackball or a touchpad provided on the housing of the mobile device 30, an external keyboard, a touchpad or a mouse, and so on.

It will be understood by those skilled in the art that the configurations illustrated in FIG. 8 and FIG. 9 as blocks are merely portions of the configurations associated with the solution according to the present disclosure, and do not constitute a limitation on the computer equipment to which the solution according to the present disclosure is applied, and that specific computer equipment can include more or fewer components than those shown in the drawings, or can combine some of the components, or can have a different arrangement of components.

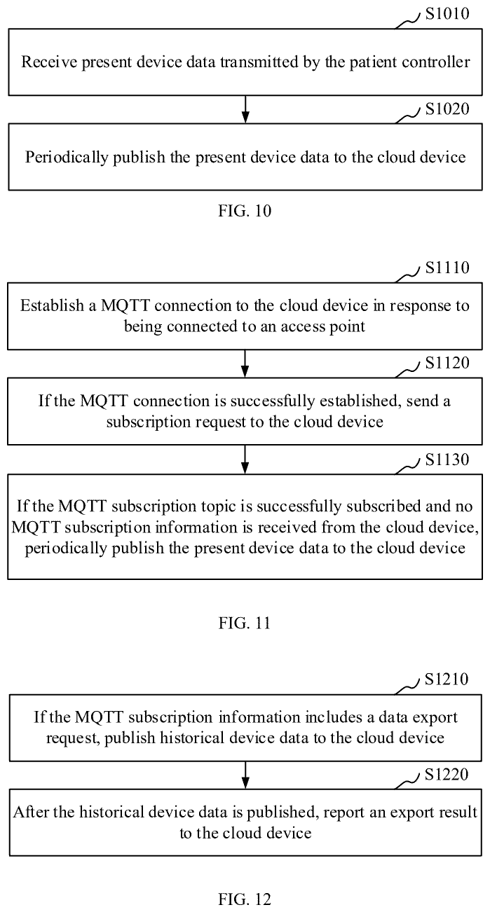

In an embodiment, as shown in FIG. 10, a method for intelligent information management of an implantable left ventricular assist device 10 applied to the communication module 110 described above is provided. The method includes the following steps.

In step S1010, present device data transmitted by the patient controller 130 is received. The present device data includes at least one of present operating data of the blood pump 120, the present power data of the implantable left ventricular assist device 10, or the device alarm message of the implantable left ventricular assist device 10.

In step S1020, the present device data is periodically published to the cloud device 20.

Specifically, the communication module 110 can communicate wirelessly with the cloud device 20. If the communication module 110 receives the present device data of the implantable left ventricular assist device 10 transmitted by the patient controller 130, the communication module 110 can periodically publish the present device data to the cloud device 20 to achieve synchronization of the present device data between the cloud device 20 and the patient controller 130. The present device data can include at least one of the present operating data of the blood pump 120, the present power data of the implantable left ventricular assist device 10, or the device alarm message of the implantable left ventricular assist device 10.

In an embodiment, the present operating data includes present start-stop state data and present pump speed data of the blood pump 120.

Specifically, the present start-stop state data of the blood pump 120 can be used to indicate that the blood pump 120 is presently in a start state or a stop state. The present pump speed data of the blood pump 120 can be used to indicate the real-time pump speed and the target pump speed of the blood pump 120.

In an embodiment, as shown in FIG. 11, the publishing the present device data periodically to the cloud device 20 includes the following steps.

In step S1110, a MQTT connection to the cloud device 20 is established in response to the communication module 110 being connected to an access point.

In step S1120, if the MQTT connection is successfully established, a subscription request is sent to the cloud device 20. The subscription request includes a MQTT subscription topic.

In step S1130, if the MQTT subscription topic is successfully subscribed and no MQTT subscription information is received from the cloud device 20, the present device data is periodically published to the cloud device 20.

It should be noted that MQTT (Message Queuing Telemetry Transport) is a lightweight messaging protocol commonly used for communication between Internet of Things (IoT) devices. Specifically, after powering on, the communication module 110 can be initialized and connected to an access point. The access point can provide wireless network access for a device, such as the communication module 110. If the communication module 110 is successfully connected to the access point, the communication module 110 can establish a MQTT connection to the cloud device 20. The cloud device 20 can be responsible for receiving messages from MQTT clients (for example, the communication module 110) and distributing them to clients that have subscribed to the corresponding topics to provide the function of a MQTT broker server. If the communication module 110 establishes a successful MQTT connection to the cloud device 20, the communication module 110 can send a subscription request to the cloud device 20. The subscription request can include a MQTT subscription topic, and the MQTT subscription topic can be a string of characters for identifying the topic or the main content of the MQTT subscription information. If the communication module 110 subscribes to the MQTT subscription topic successfully and does not receive the MQTT subscription information from the cloud device 20, the communication module 110 can periodically publish the present device data to the cloud device 20.

In some examples, the MQTT subscription topic can include SYS_INFO (system information) topic. The SYS_INFO topic can be used to periodically, proactively publishing present device data of the implantable left ventricular assist device 10, including present start-stop state data and present pump speed data of the blood pump 120, as well as the present power data and the device alarm message of the implantable left ventricular assist device 10. The present device data described above can be received by subscribing to the SYS_INFO topic. The MQTT subscription topic can also include the RESP_DATETIME (response date and time). The RESP_DATETIME topic can be used to report the setting result by the communication module 110 after completing the system time setting of the patient controller 130, for example, reporting the setting result in terms of the client ID and the corresponding setting result as the data composition for reporting the setting result. Information related to the response time of the device such as the time the device responded to the request, the timestamp of the running, etc. can be received by subscribing to the RESP_DATETIME topic.

In an embodiment, the method further includes: executing a corresponding request based on the MQTT subscription information, if the MQTT subscription topic is successfully subscribed and the MQTT subscription information is received from the cloud device 20.

Specifically, if the communication module 110 subscribes to the MQTT subscription topic successfully, and receives the MQTT subscription information from the cloud device 20, the communication module 110 can execute the corresponding request based on the MQTT subscription information. The MQTT subscription information can be the information sent by the cloud device 20 to the communication module 110 based on the corresponding request from the connected mobile device. After the communication module 110 executes the corresponding request, the cloud device 20 can accept the execution result fed back by the communication module 110 and feed the execution result to the connected mobile device of the cloud device 20.

In an embodiment, as shown in FIG. 12, the method further includes the following steps.

In step S1210, if the MQTT subscription information includes a data export request, the historical device data is published to the cloud device 20.

In step S1220, after the historical device data is published, an export result is reported to the cloud device 20.

Specifically, the cloud device 20 can send the MQTT subscription information to the communication module 110. The MQTT subscription information can for example, include a data export request, which can be a request sent by a mobile device connected to the cloud device 20 for exporting historical device data. When the MQTT subscription information received by the communication module 110 includes the data export request, then the communication module 110 can publish the historical device data to the cloud device 20. The historical device data can include historical start-stop state data and historical pump speed data of the blood pump 120, as well as historical power data and historical alarm message of the implanted left ventricular assist device 10. After the communication module 110 has finished publishing the historical device data, the communication module 110 can report the export result to the cloud device 20.

In some examples, the MQTT subscription topics can include SYS_LOG (system log) topic and the RESP_LOG (response log) topic. The SYS_LOG topic can be used to conditionally publish historical device data of the implantable left ventricular assist device 10. For example, the communication module 110 can publish historical device data only when there is a request for data export. Each time the communication module 110 publishes the historical device data, it can export the data as a 512-byte block at a time. The data composition can include a 2-byte block address and 512 bytes of historical device data. Other devices can receive the above-described historical device data by subscribing to the SYS_LOG topic. The RESP_LOG topic can be used to report the export result to the cloud device 20 after the communication module 110 completes the export of the historical device data. For example, the communication module 110 reports the export result in terms of a client ID and a corresponding export result as a data composition.

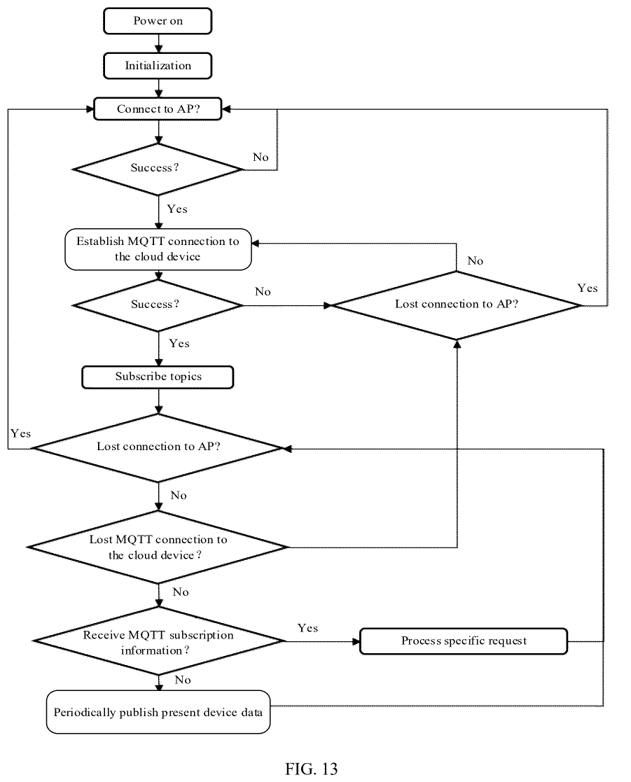

In some examples, as shown in FIG. 13, after powering on, the communication module 110 can be initialized. After the initialization of the communication module 110 is completed, the communication module 110 can be connected to the access point until the connection is successful. If the communication module 110 is successfully connected to the access point, the communication module 110 can establish a MQTT connection to the cloud device 20. If the communication module 110 establishes a successful MQTT connection to the cloud device 20, the communication module 110 can send a subscription request for the MQTT subscription topic to the cloud device 20. If the communication module 110 establishes an unsuccessful MQTT connection to the cloud device 20, the communication module 110 can determine whether the connection to the access point has been lost. If the communication module 110 loses the connection to the access point, the communication module 110 can be reconnected to the access point. If the communication module 110 remains connected to the access point, the communication module 110 can re-establish the MQTT connection to the cloud device 20. After the communication module 110 sends a subscription request for the MQTT subscription topic to the cloud device 20, the communication module 110 re-determines whether it has lost the connection to the access point. If the communication module 110 loses connection to the access point, the communication module 110 can be reconnected to the access point. If the communication module 110 remains connected to the access point, the communication module 110 can determine whether it loses the MQTT connection to the cloud device 20. If the communication module 110 loses the MQTT connection to the cloud device 20, the communication module 110 can re-determine whether it loses the connection. If the communication module 110 is still in the MQTT connection to the cloud device 20, the communication module 110 can determine whether the MQTT subscription information is received or not. If the communication module 110 receives the MQTT subscription information, the communication module 110 can execute a specific request based on the MQTT subscription information, for example, in response to a data export request to publish historical device data. If the communication module 110 does not receive the MQTT subscription information, the communication module 110 can periodically publish the present device data.

It should be understood that although the individual steps in the flow diagrams involved in the embodiments as described above are shown sequentially as indicated by the arrows, these steps are not necessarily executed sequentially in the order indicated by the arrows. Unless expressly stated herein, there is no strict order limitation on the execution of these steps, and these steps can be executed in other orders. Moreover, at least a part of the steps in the flow diagrams involved in the embodiments as described above can include a plurality of steps or phases, which are not necessarily executed at the same moment but can be executed at different times. These steps or phases are not necessarily performed sequentially but can be performed in turn or alternately with at least a part of steps or phases in other steps.

In an embodiment, there is provided a computer-readable storage medium having a computer program stored thereon. The computer program, when being executed by a processor, cause the processor to implement the steps of the method described above.

In an embodiment, there is provided a computer program product including a computer program that when executed by a processor causes the processor to implement the steps of the method described above.

A person of ordinary skill in the art can understand that realizing all or part of the processes in the methods of the above embodiments is possible by means of a computer program to instruct the relevant hardware to accomplish the same. The above-mentioned computer program can be stored in a non-volatile computer-readable storage medium, and when executed, the computer program may include processes such as the processes of the embodiments of the respective methods described above. Among other things, any reference to a memory, database, or other medium used in the embodiments provided in the present disclosure may include at least one of non-volatile or volatile memory. Non-volatile memories may include read-only memory (ROM), magnetic tape, floppy disk, flash memory, optical memory, high-density embedded non-volatile memories, resistance-resistive memory (ReRAM), magnetoresistive random access memory (MRAM), ferroelectric random access memory (FRAM), phase change memory (PCM), graphene memory and so on. The volatile memory may include a random access memory (RAM) or an external cache memory, and the like. As an illustration and not as a limitation, the RAM may be in various forms, such as static random access memory (SRAM) or dynamic random access memory (DRAM), and the like. The databases involved in the embodiments provided in the present disclosure may include at least one of a relational database or a non-relational database. The non-relational database may include a blockchain-based distributed database and the like, without limitation. The processor involved in the embodiments provided in the present disclosure may be a general-purpose processor, a central processing unit, a graphics processor, a digital signal processor, a programmable logician, a data processing logician based on quantum computing, and the like, without limitation.

The various technical features of the above-described embodiments may be combined arbitrarily, and not all possible combinations of the various technical features of the above-described embodiments have been described for the sake of conciseness of description. However, as long as there is no contradiction in the combinations of these technical features, they should be considered to be within the scope of the present specification as recorded herein.

The above-described embodiments express only several embodiments of the present disclosure, which are described in a more specific and detailed manner, but are not to be construed as a limitation of the scope of the patent disclosure. It should be pointed out that, for a person of ordinary skill in the art, several deformations and improvements can be made without departing from the conception of the present disclosure, all of which fall within the scope of protection of the present disclosure. Therefore, the scope of protection of the patent disclosure shall be subject to the attached claims.

1. A communication module configured to be connected to a patient controller of an implantable left ventricular assist device and a cloud device, the implantable left ventricular assist device further comprising a blood pump connected to the patient controller,
wherein the communication module is configured to receive present operating data of the blood pump transmitted by the patient controller, and
wherein the communication module is configured to periodically publish the present operating data to the cloud device.

2. The communication module according to claim 1, wherein the communication module is configured to receive present power data of the implantable left ventricular assist device transmitted by the patient controller, and
wherein the communication module is configured to periodically publish the present power data to the cloud device.

3. The communication module according to claim 1, wherein the communication module is configured to receive a device alarm message of the implantable left ventricular assist device transmitted by the patient controller, and
wherein the communication module is configured to periodically publish the device alarm message to the cloud device.

4. The communication module according to claim 2, comprising:
a communication unit configured to be connected to the patient controller, a power source of the implantable left ventricular assist device, and the cloud device respectively;
a display unit; and
a buck unit configured to be connected to the patient controller and the power source respectively, and also configured to be connected to the communication unit and the display unit respectively.

5. The communication module according to claim 4, comprising:
a power interface configured to be connected to the power source; and
a plurality of cables connected between the patient controller and the power interface,
wherein the plurality of cables comprise a monitoring device communication cable, a battery communication cable, a power cable, and a grounding cable,
wherein the buck unit is connected to the power cable and the grounding cable respectively, and
wherein the communication unit is connected to the monitoring device communication cable.

6. An implantable left ventricular assist system comprising:
a communication module;
an implantable left ventricular assist device comprising a patient controller, and a blood pump connected to the patient controller, wherein the patient controller is connected to the communication module, and configured to acquire present operating data of the blood pump, and transmit the acquired present operating data to the communication module;
a cloud device connected to the communication module; and
a mobile device connected to the cloud device,
wherein the communication module is configured to periodically publish the acquired present operating data to the cloud device, and
wherein the cloud device is configured to store the acquired present operating data and provide data access to the mobile device.

7. The system according to claim 6, wherein the patient controller is configured to acquire present power data of the implantable left ventricular assist device and transmit the present power data to the communication module, the communication module is configured to receive the present power data and periodically publish the present power data to the cloud device, the cloud device is configured to transmit the present power data to the mobile device in response to receiving the present power data, and the mobile device is configured to output a corresponding prompt message in response to receiving the present power data.

8. The system according to claim 6, wherein the communication module is configured to receive a device alarm message of the implantable left ventricular assist device transmitted by the patient controller and periodically publish the device alarm message to the cloud device, the cloud device is configured to transmit the device alarm message to the mobile device in response to receiving the device alarm message, and the mobile device is configured to output a corresponding prompt message in response to receiving the device alarm message.

9. The system according to claim 6, wherein the communication module comprises:
a communication unit connected to the patient controller and the cloud device, respectively, and further connected to a power source of the implantable left ventricular assist device;
a display unit; and
a buck unit connected to the patient controller, the communication unit, and the display unit, respectively, and further connected to the power source.

10. The system according to claim 9, wherein the communication module comprises:
a power interface configured to be connected to the power source; and
a plurality of cables connected between the patient controller and the power interface,
wherein the plurality of cables comprise a monitoring device communication cable, a battery communication cable, a power cable, and a grounding cable,
wherein the buck unit is connected to the power cable and the grounding cable, respectively, and
wherein the communication unit is connected to the monitoring device communication cable.

11. A method for intelligent information management of an implantable left ventricular assist device, the method being applied to a communication module, configured to be connected to a patient controller of an implantable left ventricular assist device and a cloud device, the implantable left ventricular assist device further comprising a blood pump connected to the patient controller, and the method comprising:
receiving, by the communication module, present device data transmitted by the patient controller, wherein the present device data comprises at least one of the present operating data of the blood pump, the present power data of the implantable left ventricular assist device, or the device alarm message of the implantable left ventricular assist device; and
publishing, by the communication module, the present device data to the cloud device periodically.

12. The method according to claim 11, wherein the present operating data comprises present start-stop state data and present pump speed data of the blood pump.

13. The method according to claim 11, wherein the publishing, by the communication module, the present device data to the cloud device periodically comprises:
establishing, by the communication module, a MQTT connection to the cloud device in response to the communication module being connected to an access point;
sending, by the communication module, a subscription request to the cloud device if the MQTT connection is successfully established, wherein the subscription request comprises a MQTT subscription topic; and
publishing, by the communication module, the present device data to the cloud device periodically if the MQTT subscription topic is successfully subscribed and no MQTT subscription information is received from the cloud device.

14. The method according to claim 13, further comprising:
executing, by the communication module, a corresponding request based on the MQTT subscription information, if the MQTT subscription topic is successfully subscribed and the MQTT subscription information is received from the cloud device.

15. The method according to claim 14, further comprising:
publishing, by the communication module, historical device data to the cloud device if the MQTT subscription information comprises a data export request; and
reporting, by the communication module, an export result to the cloud device after the historical device data is published.

16. The method according to claim 11, wherein the communication module comprises:
a communication unit configured to be connected to the patient controller, a power source of the implantable left ventricular assist device, and the cloud device respectively;
a display unit; and
a buck unit configured to be connected to the patient controller and the power source respectively, and also configured to be connected to the communication unit and the display unit respectively.

17. The method according to claim 16, wherein the communication module comprises:
a power interface configured to be connected to the power source; and
a plurality of cables connected between the patient controller and the power interface,
the plurality of cables comprising a monitoring device communication cable, a battery communication cable, a power cable, and a grounding cable,
the buck unit being connected to the power cable and the grounding cable respectively, and the communication unit being connected to the monitoring device communication cable.

18. The method according to claim 13, wherein the communication module comprises:
a communication unit configured to be connected to the patient controller, a power source of the implantable left ventricular assist device, and the cloud device respectively;
a display unit; and
a buck unit configured to be connected to the patient controller and the power source respectively, and also configured to be connected to the communication unit and the display unit respectively.

19. The method according to claim 18, wherein the communication module comprises:
a power interface configured to be connected to the power source; and
a plurality of cables connected between the patient controller and the power interface,
the plurality of cables comprising a monitoring device communication cable, a battery communication cable, a power cable, and a grounding cable,
the buck unit being connected to the power cable and the grounding cable respectively, and
the communication unit being connected to the monitoring device communication cable.

20. The method according to claim 14, wherein the communication module comprises:
a communication unit configured to be connected to the patient controller, a power source of the implantable left ventricular assist device, and the cloud device respectively;
a display unit; and
a buck unit configured to be connected to the patient controller and the power source respectively, and also configured to be connected to the communication unit and the display unit respectively.
